(19) United States
(12) Patent Application Publication
(10) Pub. No.: US 2024/0019432 A1
(43) Pub. Date: Jan. 18, 2024

(54) DIAGNOSTIC METHOD FOR MULTIPLE SCLEROSIS

(71) Applicant: Belgian Volition SRL (BE/BE), Isnes (BE)

(72) Inventors: Jason Bradley TERRELL, Isnes (BE); Jacob Vincent MICALLEF, Isnes (BE); Mark Edward ECCLESTON, Isnes (BE); Marielle HERZOG, Isnes (BE)

(21) Appl. No.: 18/256,191

(22) PCT Filed: Dec. 8, 2021
(86) PCT No.: PCT/EP2021/084718

§ 371 Date: Jun. 6, 2023

Related U.S. Application Data
(60) Provisional application No. 63/122,866, filed on Dec. 8, 2020.

(51) Int. Cl. G01N 33/564

(52) U.S. Cl. CPC G01N 33/564

(57) ABSTRACT

The invention relates methods for diagnosing patients with multiple sclerosis by detecting cell free nucleosomes as biomarkers in body fluid samples.

### FIELD OF THE INVENTION

The present invention relates to cell free nucleosomes as biomarkers in body fluid samples for patients with multiple sclerosis.

### BACKGROUND OF THE INVENTION

Multiple sclerosis (MS) is a chronic neurodegenerative disease of the brain and spinal cord involving a loss of the myelin sheath that surrounds and protects the nerves. Symptoms are non-specific but often include fatigue and problems with vision, mobility, sensation and balance. More than 300,000 people are diagnosed with MSA in the USA. The disease is more common in women than men and is typically diagnosed in people aged 20-30 years, although older people may also be diagnosed.

The underlying cause of MS is still unknown but is thought to include genetic and environmental factors. The symptoms of the disease are caused by patches of autoimmune damage to the myelin sheath which may become inflamed and disrupt or prevent normal nerve signal transmission. Over time the inflamed patches may lead to scarring, or sclerosis, of the myelin sheath and repeated attacks can lead to permanent damage to the underlying nerves.

Once diagnosed, MS is a lifelong condition that can cause disability but is not normally terminal. Patients are classified into 3 main groups. Most patients with MS have a relapsing-remitting disease course where there are periods of new symptoms or relapses that develop over days or weeks (sometimes referred to as an attack or flare-up), followed by quiet periods of disease remission that can last months or even years. There is no cure currently available, although treatments are available to manage symptoms and increase the recovery from attacks. As the disease course continues, patients with relapsing-remitting MS may experience a more continuous increase in their disability, with fewer or no relapses, known as secondary progressive MS. Some 10-15% of MS patients are diagnosed with primary progressive MS in which disability increases from the beginning and relapses are rare. The rate of progression of disability can vary widely between patients.

Due to the range of symptoms, MS can be difficult to successfully diagnose. Current tests focus on investigating evidence of nerve damage, such as a neurological examination or magnetic resonance imaging (MRI) scan. Lumbar punctures are also used to take a sample of the cerebrospinal fluid (CSF) and test for the presence of immune cells or oligoclonal bands of antibodies associated with the autoimmune response.

A simple blood test for MS would be less invasive than lumbar puncture and have cost advantages over both MRI scanning and lumbar puncture. Repeated lumbar puncture for monitoring purposes and management of the disease is clearly undesirable and repeated MRI scanning is expensive. There remains a need in the art to provide simple, non-invasive, cost-effective methods to identify patients with multiple sclerosis, especially for ongoing management of the disease and to detect patients more at risk of suffering a relapse.

### SUMMARY OF THE INVENTION

According to a first aspect, there is provided a method of diagnosing multiple sclerosis in a subject, comprising contacting a body fluid sample obtained from the subject with a binding agent to detect or measure the level of cell free nucleosomes or a component thereof.

According to a further aspect, there is provided a method of detecting a subject in need of medical treatment for multiple sclerosis, comprising:
(i) contacting a body fluid sample obtained from the subject with a binding agent to detect or measure the level of cell free nucleosomes or a component thereof; and
(ii) using the level of cell free nucleosomes as an indicator that the subject is in need of medical treatment for multiple sclerosis.

According to a further aspect, there is provided a use of a cell free nucleosome comprising histone H3.1 as a biomarker in a body fluid sample for the diagnosis of multiple sclerosis.

According to a further aspect, there is provided a method of treating a subject suffering from multiple sclerosis, comprising:
(i) contacting a body fluid sample obtained from the subject with a binding agent to measure the level of cell free nucleosomes or a component thereof;
(ii) using the level of cell free nucleosomes or component thereof measured in step (i) as indicative of the presence and/or severity of multiple sclerosis in the subject; and
(iii) administering a treatment to the subject identified in need of treatment in step (ii).

### BRIEF DESCRIPTION OF FIGURES

FIG. 1. Box plot and ROC curve (receiver operating characteristic curve) for measured levels of nucleosomes containing histone isoform H3.1 in patients diagnosed with MS and in healthy subjects.

FIG. 2. Box plot and ROC curve for measured levels of nucleosomes containing histone PTM H3K27Me3 in patients diagnosed with MS and in healthy subjects.

FIG. 3. Box plot and ROC curve for measured levels of nucleosomes containing histone PTM H3R2,R8,R17Cit in patients diagnosed with MS and in healthy subjects.

### DETAILED DESCRIPTION

The present invention utilises elevated levels of cell free nucleosomes to identify subjects with multiple sclerosis.

Therefore, according to a first aspect, there is provided a method of diagnosing multiple sclerosis in a subject, comprising contacting a body fluid sample obtained from the subject with a binding agent to detect or measure the level of cell free nucleosomes or a component thereof.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, Methods Mol. Biol. (2007) 361: 25-62).

References to “nucleosome” may refer to “cell free nucleosome” when detected in body fluid samples. It will be appreciated that the term cell free nucleosome throughout this document is intended to include any cell free chromatin fragment that includes one or more nucleosomes.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or DNA.

Methods and uses of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells.

Normal cell turnover in adult humans involves the creation by cell division of some 10¹¹ cells daily and the death of a similar number, mainly by apoptosis. During the process of apoptosis chromatin is broken down into mononucleosomes and oligonucleosomes which are released from the cells. Under normal conditions the levels of circulating nucleosomes found in healthy subjects is reported to be low. Elevated levels are found in subjects with a variety of conditions including many cancers, auto-immune diseases, inflammatory conditions, stroke and myocardial infarction (Holdenreider & Stieber, Crit. Rev. Clin. Lab. Sci. (2009) 46(1): 1-24).

Previous nucleosome ELISA methods were used primarily in cell culture, usually as a method to detect apoptosis (Salgame et al., Nucleic Acids Res. (1997) 25(3): 680-681; Holdenrieder et al. Clin. Chem. Lab. Med. (2001) 39(7): 596-605; van Nieuwenhuijze et al., Ann. Rheum. Dis. (2003) 62: 10-14), but are also used for the measurement of circulating cell free nucleosomes in serum and plasma (Holdenrieder et al. (2001)). Cell free serum and plasma nucleosome levels released into the circulation by dying cells have been measured by ELISA methods in studies of a number of different cancers to evaluate their use as a potential biomarker.

The cell free nucleosome may be mononucleosomes, oligonucleosomes, a constituent part of a larger chromatin fragment or a constituent part of a NET or a mixture thereof.

Mononucleosomes and oligonucleosomes can be detected by Enzyme-Linked ImmunoSorbant Assay (ELISA) and several methods have been reported (e.g. Salgame et al. (1997); Holdenrieder et al. (2001); van Nieuwenhuijze et al. (2003)). These assays typically employ an anti-histone antibody (for example anti-H2B, anti-H3 or anti-H1, H2A, H2B, H3 and H4) as capture antibody and an anti-DNA or anti-H2A-H2B-DNA complex antibody as detection antibody.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts. Assays for these types of chromatin fragments are known in the art (for example, see WO 2005/019826, WO 2013/030579, WO 2013/030578, WO 2013/084002 which are herein incorporated by reference).

In one embodiment, the component of the cell free nucleosome comprises an epigenetic feature of the cell free nucleosome.

The biomarker used in the methods of the invention may be the level of cell free nucleosomes per se and/or an epigenetic feature of a cell free nucleosome. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of: a post-translational histone modification, a histone isoform, a modified nucleotide and/or proteins bound to a nucleosome in a nucleosome-protein adduct.

In one embodiment, the epigenetic feature of the nucleosome comprises one or more histone variants or isoforms. The epigenetic feature of the cell free nucleosome may be a histone isoform, such as a histone isoform of a core nucleosome, in particular a histone H3 isoform. The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. They can be classed into a number of families which are subdivided into individual types. The nucleotide sequences of a large number of histone variants are known and publicly available for example in the National Human Genome Research Institute NHGRI Histone Database (Mariño-Ramírez et al. The Histone Database: an integrated resource for histones and histone fold-containing proteins. Database Vol. 2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, variants of histone H2 include H2A1, H2A2, mH2A1, mH2A2, H2AX and H2AZ. In another example, histone isoforms of H3 include H3.1, H3.2 and H3t.

In one embodiment, the histone isoform is H3.1. As shown by the Example presented herein, the level of H3.1 was surprisingly elevated in patients with multiple sclerosis. Therefore, according to another aspect of the invention, there is provided the use of a cell free nucleosome comprising histone H3.1 as a biomarker in a body fluid sample for the diagnosis of multiple sclerosis.

The structure of nucleosomes can vary by post translational modification (PTM) of histone proteins. PTM of histone proteins typically occurs on the tails of the core histones and common modifications include acetylation, methylation or ubiquitination of lysine residues as well as methylation of arginine residues and phosphorylation of serine residues and many others. Many histone modifications are known in the art and the number is increasing as new modifications are identified (Zhao and Garcia (2015) Cold Spring Harb Perspect Biol, 7: a025064). Therefore, in one embodiment, the epigenetic feature of the cell free nucleosome may be a histone post translational modification (PTM). The histone PTM may be a histone PTM of a core nucleosome, e.g. H3, H2A, H2B or H4, in particular H3, H2A or H2B. In particular, the histone PTM is a histone H3 PTM. Examples of such PTMs are described in WO 2005/019826.

For example, the post translational modification may include acetylation, methylation, which may be mono-, di-or tri-methylation, phosphorylation, ribosylation, citrullination, ubiquitination, hydroxylation, glycosylation, nitrosylation, glutamination and/or isomerisation (see Ausio (2001) Biochem Cell Bio 79: 693). In one embodiment, the histone PTM is selected from citrullination or methylation. In a further embodiment, the histone PTM is H3 citrulline (H3cit) or H4 citrulline (H4cit). In a yet further embodiment, the histone PTM is H3R2,R8,R17Cit. In another embodiment, the histone PTM is H3 methylation, such as H3K27Me3.

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes without limitation, anti-pan-acetylation antibodies, anti-pan-citrullination antibodies (e.g. a pan-citrullination H3 antibody such as an H3R2,R8,R17Cit antibody]) or anti-ubiquitin antibodies.

In one embodiment, the epigenetic feature of the nucleosome comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues (or 5-hydroxymethylcytosine residues or other nucleotides or modified nucleotides) than other nucleosomes. In one embodiment, the DNA modification is selected from 5-methylcytosine or 5-hydroxymethylcytosine.

In one embodiment, the epigenetic feature of the nucleosome comprises one or more protein-nucleosome adducts or complexes. A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art.

In one embodiment, the protein adducted to the nucleosome (and which therefore may be used as a biomarker) is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate. All of the circulating nucleosomes and nucleosome moieties, types or subgroups described herein may be useful in the present invention.

It will be understood that more than one epigenetic feature of cell free nucleosomes may be detected in methods and uses of the invention. Multiple biomarkers may be used as a combined biomarker. Therefore, in one embodiment, the use comprises more than one epigenetic feature of cell free nucleosomes as a combined biomarker. The epigenetic features may be the same type (e.g. PTMs, histone isoforms, nucleotides or protein adducts) or different types (e.g. a PTM in combination with a histone isoform). For example, a post-translational histone modification and a histone variant may be detected (i.e. more than one type of epigenetic feature is detected). Alternatively, or additionally, more than one type of post-translational histone modification is detected, or more than one type of histone isoform is detected. In one aspect, the use comprises a post-translational histone modification and a histone isoform as a combined biomarker in a sample, for the diagnosis or detection of multiple sclerosis. In one embodiment, the combined biomarker is H3.1 and citrullinated H3 (such as H3R2,R8,R17Cit) and/or methylated H3 (such as H3K27Me).

The term “biomarker” means a distinctive biological or biologically derived indicator of a process, event, or condition. Biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

Methods and uses described herein may be tested in body fluid samples, in particular CSF, blood, serum or plasma samples. Preferably, plasma samples are used. Plasma samples may be collected in collection tubes containing one or more anticoagulants such as ethylenediamine tetraacetic acid (EDTA), heparin, or sodium citrate, in particular EDTA.

### Diagnosis and Monitoring Methods

According to a further aspect, there is provided a method of monitoring multiple sclerosis in a subject, comprising:
(i) contacting a body fluid sample obtained from the subject with a binding agent to detect or measure the level of cell free nucleosomes or a component thereof;
(ii) repeating the detection or measurement of the level of cell free nucleosomes or a component thereof in a body fluid obtained from the subject on one or more occasions;
(iii) using any changes in the level of cell free nucleosomes or component thereof to monitor multiple sclerosis in the subject.

Such methods can be used to monitor the progression or progress of the disease in a subject to determine whether medical intervention is required. For example, if a subject is determined to have a mild stage of disease or is in remission, then the invention may be used for the purposes of monitoring disease progression for future development of a relapse. For example, if the method comprises a sample from a subject determined to have a mild stage of disease, then the biomarker level measurements can be repeated at another time point to establish if the biomarker level has changed.

Detecting and/or quantifying may be performed directly on the body fluid nucleosome sample, or on a purified or enriched nucleosome sample, or indirectly on an extract therefrom, or on a dilution thereof. Quantifying the amount of the biomarker present in a sample may include determining the concentration of the biomarker present in the sample. Uses and methods of detecting, monitoring and of diagnosis according to the invention described herein are useful to confirm the existence of a disease, to monitor development of the disease by assessing onset and progression, or to assess amelioration or regression of the disease. Uses and methods of detecting, monitoring and of diagnosis are also useful in methods for assessment of clinical screening, prognosis, choice of therapy, evaluation of therapeutic benefit, i.e. for drug screening and drug development.

The detection or measurement may comprise an immunoassay, immunochemical, mass spectroscopy, chromatographic, chromatin immunoprecipitation or biosensor method. In particular, detection and/or measurement may comprise a 2-site immunoassay method for nucleosome moieties. Such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. Alternatively, the immunoassay may employ two anti-nucleosome binding agents which are both directed to bind to epigenetic features of a nucleosome. Also, detection and/or measurement may comprise a 2-site immunoassay, for example employing combinations of a labelled or immobilized: anti-nucleosome, anti-histone modification, anti-histone variant/isoform, anti-DNA modification or anti-adducted protein binding agent.

In one embodiment, the method of detection or measurement comprises contacting the body fluid sample with a solid phase comprising a binding agent that detects cell free nucleosomes or a component thereof, and detecting binding to said binding agent.

In one embodiment, the method of detection or measurement comprises: (i) contacting the sample with a first binding agent which binds to an epigenetic feature of a cell free nucleosome; (ii) contacting the sample bound by the first binding agent in step (i) with a second binding agent which binds to cell free nucleosomes; and (iii) detecting or quantifying the binding of the second binding agent in the sample.

In another embodiment, the method of detection or measurement comprises: (i) contacting the sample with a first binding agent which binds to cell free nucleosomes; (ii) contacting the sample bound by the first binding agent in step (i) with a second binding agent which binds to an epigenetic feature of the cell free nucleosome; and (iii) detecting or quantifying the binding of the second binding agent in the sample.

Detecting or measuring the level of the biomarker(s) may be performed using one or more reagents, such as a suitable binding agent. For example, the one or more binding agents may comprise a ligand or binder specific for the desired biomarker, e.g. nucleosomes or component part thereof, an epigenetic feature of a nucleosome, a structural/shape mimic of the nucleosome or component part thereof, optionally in combination with one or more interleukins.

It will be clear to those skilled in the art that the terms “antibody”, “binder” or “ligand” as used herein are not limiting but are intended to include any binder capable of binding to particular molecules or entities and that any suitable binder can be used in the method of the invention. It will also be clear that the term “nucleosomes” is intended to include mononucleosomes, oligonucleosomes and any protein-DNA chromatin fragments that can be analysed in fluid media.

Methods of detecting biomarkers are known in the art. The reagents may comprise one or more ligands or binders, for example, naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

The term “detecting” or “diagnosing” as used herein encompasses identification, confirmation, and/or characterisation of a disease state and use as an aid to diagnosis where the detection or diagnostic method is used alongside other methods to produce a more complete assessment of a patients' condition. Methods of detecting, monitoring and of diagnosis according to the invention are useful to confirm the existence of a disease, to monitor development of the disease by assessing onset and progression, to predict the prognosis of the disease, to predict and/or monitor and/or manage disease relapse, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

Methods of the invention may involve normalisation of marker levels. For example, the level of cell free nucleosomes containing a particular epigenetic feature may be normalised against the level of nucleosomes per se (or some other type of nucleosomes or parameter) to express the level as a proportion of nucleosomes containing the feature. For example, to express the level of citrullinated nucleosomes as the proportion of nucleosomes that are citrullinated.

In one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission. Therefore, in one embodiment, the method is repeated on one or more occasions and any changes in the level of cell free nucleosomes or component thereof is used to monitor the progression of multiple sclerosis in the subject.

In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the nature or amount of the biomarker(s) in test samples taken on different occasions.

A change in the level of the biomarker in the test sample relative to the level in a previous test sample taken earlier from the same test subject may be indicative of a beneficial effect, e.g. stabilisation or improvement, of said therapy on the disorder or suspected disorder. Furthermore, once treatment has been completed, the method of the invention may be periodically repeated in order to monitor for the recurrence or relapse of a disease.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

In a further embodiment the monitoring of more rapid changes due to fast acting therapies may be conducted at shorter intervals of hours or days.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

A further aspect of the invention is a kit for diagnosing or monitoring the progression of multiple sclerosis, comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

Suitably, biosensors for detection of one or more biomarkers combine biomolecular recognition with appropriate means to convert detection of the presence, or quantitation, of the biomarker in the sample into a signal. Biosensors can be adapted for “alternate site” diagnostic testing, e.g. in the ward, outsubjects' department, surgery, home, field and workplace. Biosensors to detect one or more biomarkers of the invention include lateral flow, acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers.

Biomarkers for detecting the presence of a disease are essential targets for discovery of novel targets and drug molecules that retard or halt progression of the disorder. As the level of the biomarker is indicative of disorder and of drug response, the biomarker is useful for identification of novel therapeutic compounds in in vitro and/or in vivo assays. Biomarkers described herein can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The immunoassays described herein include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels. All of said immunoassay methods are well known in the art, see for example Salgame et al. (1997) and van Nieuwenhuijze et al. (2003).

Identifying, detecting and/or quantifying can be performed by any method suitable to identify the presence and/or amount of a specific protein in a biological sample from a subject or a purification or extract of a biological sample or a dilution thereof. In particular, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner. The present invention finds particular use in plasma samples which may be obtained from the subject.

Identification, detection and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed by one or more method(s) selected from the group consisting of: immunoassay, immunochromatography, SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving detection and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

The identification of biomarkers for a disease state permits integration of diagnostic procedures and therapeutic regimes. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

References to “subject” or “patient” are used interchangeably herein. The subject may be a human or an animal subject. In one embodiment, the subject is a human. In one embodiment, the subject is a (non-human) animal. The panels and methods described herein may be performed in vitro, in vivo or ex vivo.

Detecting and/or quantifying may be compared to a cut-off level. Cut-off values can be predetermined by analysing results from multiple patients and controls, and determining a suitable value for classifying a subject as with or without the disease. For example, for diseases where the level of biomarker is higher in patients suffering from the disease, then if the level detected is higher than the cut-off, the patient is indicated to suffer from the disease. Alternatively, for diseases where the level of biomarker is lower in patients suffering from the disease, then if the level detected is lower than the cut-off, the patient is indicated to suffer from the disease. The advantages of using simple cut-off values include the ease with which clinicians are able to understand the test and the elimination of any need for software or other aids in the interpretation of the test results. Cut-off levels can be determined using methods in the art.

Detecting and/or quantifying may also be compared to a control. It will be clear to those skilled in the art that the control subjects may be selected on a variety of basis which may include, for example, subjects known to be free of the disease or may be subjects with a different disease (for example, for the investigation of differential diagnosis). The “control” may comprise a healthy subject, a non-diseased subject and/or a subject in remission. The control may also be a subject with multiple sclerosis displaying no, or mild, symptoms. Mild symptoms may include manageable symptoms which do not require hospital intervention and/or invasive medical treatment.

Comparison with a control is well known in the field of diagnostics. The range of values found in the control group may be used as a normal or healthy or reference range against which the values found for test subjects can be compared. For example, if the reference range is <10 units, then a test value of 5 units would be considered normal, or not in need of treatment, but a value of 11 units would be considered abnormal and indicative of a need for treatment.

Therefore, in one embodiment, the method additionally comprises comparing the level of cell free nucleosomes or component thereof in the body fluid sample of the subject with one or more controls. For example, the method may comprise comparing the level of cell free nucleosomes present in a sample obtained from the subject with the level of cell free nucleosomes present in a sample obtained from a normal subject. The control may be a healthy subject.

In one embodiment, the level of cell free nucleosomes or component thereof is elevated compared to a control.

It will be understood that it is not necessary to measure controls levels for comparative purposes on every occasion. For example, for healthy/non-diseased controls, once the ‘normal range’ is established it can be used as a benchmark for all subsequent tests. A normal range can be established by obtaining samples from multiple control subjects without the disease and testing for the level of biomarker. Results (i.e. biomarker levels) for subjects suspected to have the disease can then be examined to see if they fall within, or outside of, the respective normal range. Use of a ‘normal range’ is standard practice for the detection of disease.

In one embodiment, the method additionally comprises determining at least one clinical parameter for the patient. This parameter can be used in the interpretation of results. Clinical parameters may include any relevant clinical information for example, without limitation, body temperature, gender, weight, Body Mass Index (BMI), smoking status and dietary habits. Therefore, in one embodiment, the clinical parameter is selected from the group consisting of: body temperature, age, sex and body mass index (BMI).

According to another aspect of the invention, there is provided the use of a binding agent in the manufacture of a kit for use in a method of diagnosing or monitoring multiple sclerosis, comprising:
(i) contacting a body fluid sample obtained from the subject with the binding agent to detect or measure the level of cell free nucleosomes or a component thereof; and
(ii) using the level of cell free nucleosomes detected to diagnose or monitor the subject.

According to another aspect of the invention, there is provided a method of detecting a subject in need of medical treatment for multiple sclerosis, comprising:
(i) contacting a body fluid sample obtained from the subject with a binding agent to detect or measure the level of cell free nucleosomes or a component thereof; and
(ii) using the level of cell free nucleosomes as an indicator that the subject is in need of medical treatment for multiple sclerosis.

### Additional Biomarkers

The level of cell free nucleosomes may be detected or measured as one of a panel of measurements. The panel may comprise different epigenetic features of the nucleosome as described hereinbefore (e.g. a histone isoform and a PTM). Biomarkers useful in a panel test for the detection of multiple sclerosis may include, without limitation, oligoclonal immunoglobulin G (IgG) or immunoglobin M (IgM) proteins, cytokine moieties (particularly interleukins), C-reactive protein, D-Dimer, factor VII-activating protease (FSAP), fibrinogen and fibrin/fibrinogen breakdown products. In one embodiment, the panel comprises one or more cytokines, such as one or more interleukins. In preferred embodiments the panel measurement is made in a CSF, blood, plasma or serum sample.

Interleukins (ILs) are a group of cytokines, usually secreted by leukocytes, that act as signal molecules. They have key roles in stimulating immune responses and inflammation. They were first identified in the 1970s and have been designated numerically as more interleukin types have been discovered. Examples of interleukins include, but are not limited to: IL-1, IL-2, IL-3, IL-4, IL-5, IL-6, IL-7, IL-8, IL-9, IL-10, IL-11, IL-12, IL-13, IL-14 and IL-15.

In one embodiment, the one or more interleukins is selected from the group consisting of: Interleukin-6 (IL-6) and Interleukin-12 (IL-12).

The interleukin may be IL-6. Interleukin-6 (IL-6) is a cytokine with a wide variety of biological functions. It is a potent inducer of fever and the acute phase response. The sequence of human IL-6 is known in the art and is described at UniProt Accession No. P05231. In one particular embodiment, the interleukin may be IL-6.

Alternatively, or additionally, the interleukin may be IL-12. Interleukin-12 (IL-12) is a T cell stimulating factor because it stimulates the growth and function of T cells. It is a heterodimeric cytokine comprised of IL-12A and IL-12B. The sequence of human IL-12A is known in the art and is described at UniProt Accession No. P29459 and the sequence of human IL-12B is also known and described at UniProt Accession No. P29460. In one particular embodiment, the interleukin may be IL-12.

In one embodiment, the panel comprises a cell free nucleosome or an epigenetic feature thereof and an interleukin. In another embodiment, the panel comprises an epigenetic feature of a cell free nucleosome and two interleukins. For example, the cell free nucleosome measurement can be combined with more than one interleukin measurement, such as IL-6 and IL-12. In a further embodiment, the epigenetic feature of a cell free nucleosome is selected from a histone isoform, such as H3.1, and a post translationally modified histone, such as H3 citrullinated and/or H3 methylated. In a yet further embodiment, the panel of measurements is H3.1, H3R2, R8,R17Cit and H3K27Me3.

Models can be derived using the biomarkers of the invention. Methods for deriving models or algorithms are well known in the art and suitable software packages are available. Typical software tools for this purpose include SPSS (Statistical Package for the Social Sciences) and “R”. These software packages provide for linear and non-linear data modelling of clinical data.

It will be clear to those skilled in the art, that any combination of the biomarkers disclosed herein may be used in panels and algorithms for the detection of multiple sclerosis, and that further markers may be added to a panel including these markers.

According to an aspect of the invention there is provided the use of a panel test to detect a patient with multiple sclerosis, wherein the panel test comprises reagents to detect measurements of nucleosomes or a component thereof and one or more interleukins, in a sample obtained from the patient.

### Methods of Treatment

According to a further aspect, there is provided a method of treating a subject suffering from multiple sclerosis, comprising:
(i) contacting a body fluid sample obtained from the subject with a binding agent to measure the level of cell free nucleosomes or a component thereof;
(ii) using the level of cell free nucleosomes or component thereof measured in step (i) as indicative of the presence and/or severity of multiple sclerosis in the subject; and
(iii) administering a treatment to the subject identified in need of treatment in step (ii).

According to a further aspect, there is provided a method of treating multiple sclerosis in a subject in need thereof, which comprises the step of administering a therapeutic agent to a subject identified as having differing levels of cell free nucleosomes in a sample obtained from said subject, when compared to the level of cell free nucleosomes in a sample obtained from a control subject.

Step (iii) may comprise administering a therapeutic agent. The treatment may be focused on treating relapses of MS symptoms (for example, with steroid medicine), treating specific MS symptoms and/or treatment to reduce the number of relapses (i.e. disease-modifying therapies). In one embodiment, the treatment is selected from one or more of: steroids (for example corticosteroids, such as prednisone and methylprednisolone), interferon beta medications (such as Avonex, Betaferon, Extavia, Plegridy and Rebif), glatiramer acetate, fingolimod, dimethyl fumarate, diroximel fumarate, teriflunomide, siponimod, cladribine, monoclonal antibody therapies (such as Ocrelizumab, Natalizumab and Alemtuzumab) or haematopoietic stem cell transplantation (HSCT).

According to another aspect of the invention there is provided a method of treating multiple sclerosis comprising identifying a patient in need of treatment for multiple sclerosis using a panel test and providing said treatment, wherein the panel test comprises reagents to detect measurements of nucleosomes or components thereof. A patient with multiple sclerosis is expected to have a higher level of cell free nucleosomes compared to a control.

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLE 1

Plasma samples were collected from 24 patients diagnosed with MS and from 19 healthy subjects for nucleosome assay. Whole blood samples were collected into plasma blood collection tubes and were centrifuged within 2 hours of venepuncture. The plasma was transferred into cryovials and stored frozen until assayed. Stored plasma samples were thawed and centrifuged for 2 minutes at 14000 g prior to assay.

Measurements for 3 different nucleosome types were performed on the samples. These were nucleosomes containing histone variant H3.1 (H3.1-nucleosomes), nucleosomes containing the histone PTM H3K27Me3 (histone H3 methylated at lysine residue 27) and nucleosomes containing the histone PTM H3R2,R8,R17Cit (histone H3 citrullinated at arginine residues 2, 8 and 17). Measurements for nucleosomes were performed by immunoassay using an automated immunoassay instrument. Briefly, calibrant or sample (50 μl) was incubated with an acridinium ester labelled anti-nucleosome antibody (50 μl) and assay buffer (100 μl ) for 1800 seconds at 37° C. Magnetic beads coated with an anti-H3.1, anti-H3K27Me3 or anti-H3R2,R8,R17Cit antibody (20 μl) were added and the mixture was incubated a further 900 seconds. The magnetic beads were then isolated, washed 3 times and magnetic bound acridinium ester was determined by luminescence output over 7000 milliseconds. Results were interpolated from a standard curve.

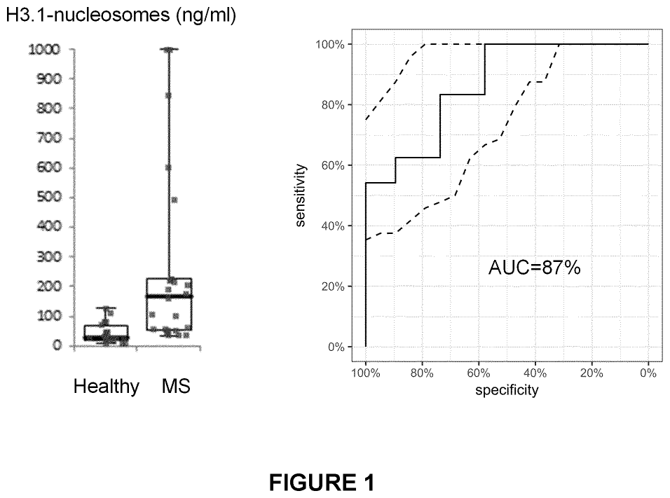

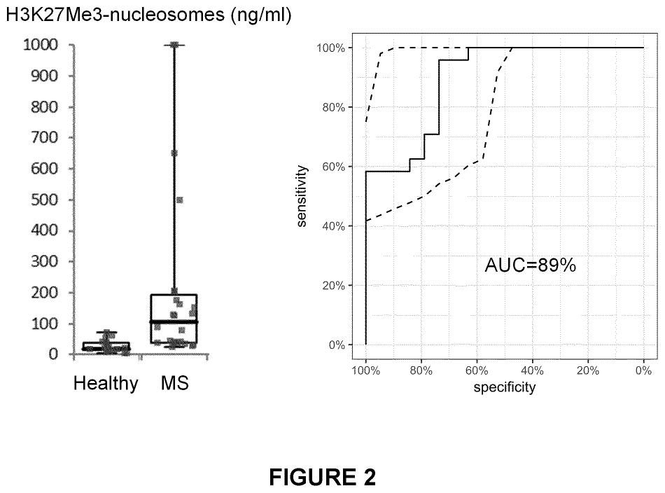

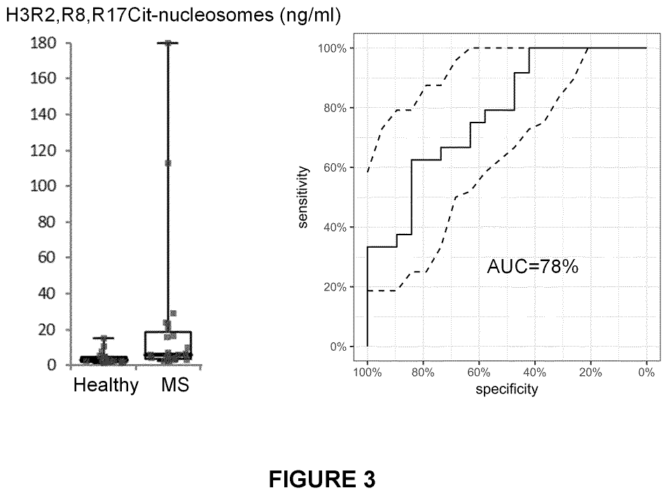

The results showed that plasma levels of the three nucleosomes types were elevated in MS patients, although with different patterns. H3.1-nucleosome levels were elevated in MS and the results produced a ROC curve with an area under the curve (AUC) of 87% and a sensitivity of 54% at 100% specificity (FIG. 1). H3K27Me3-nucleosome levels were elevated in MS and the results produced a ROC curve with an area under the curve (AUC) of 89% and a sensitivity of 58% at 100% specificity (FIG. 2). H3R2,R8,R17Cit-nucleosome levels were elevated in MS and the results produced a ROC curve with an area under the curve (AUC) of 78% and a sensitivity of 33% at 100% specificity (FIG. 3).

We concluded that nucleosome levels are elevated in the body fluids of MS patients and that nucleosome measurements are an effective indicator of the presence and severity of disease and may provide an indicator of the likelihood of, and/or likely severity of, a relapse or attack, as well as an indicator of the amelioration of symptoms or an attack.

1. A method of diagnosing multiple sclerosis in a subject, comprising: contacting a body fluid sample obtained from the subject with a binding agent to detect or measure a level of cell free nucleosomes or a component thereof.

2. The method of claim 1, wherein the body fluid sample is a blood, serum or plasma sample.

3. The method of claim 1, wherein the body fluid sample is a cerebrospinal fluid sample.

4. The method of claim 1, wherein the component of the cell free nucleosome comprises an epigenetic feature of the cell free nucleosome.

5. The method of claim 4, wherein the epigenetic feature is a histone isoform, such as a histone isoform of a core nucleosome, in particular a histone H3 isoform.

6. The method of claim 5, wherein the histone isoform is H3.1.

7. The method of claim 4, wherein the epigenetic feature is a histone post translational modification (PTM).

8. The method of claim 7, wherein the histone PTM is selected from citrullination or methylation.

9. The method claim 1, wherein the level of cell free nucleosomes or component thereof is detected or measured using a technique selected from the group consisting of immunoassay, immunochemical, mass spectroscopy, chromatographic, chromatin immunoprecipitation and biosensor.

10. The method of claim 1, wherein the method of detection or measurement comprises contacting the body fluid sample with a solid phase comprising a binding agent that detects cell free nucleosomes or a component thereof, and detecting binding to said binding agent.

11. The method of claim 1, wherein the method of detection or measurement comprises: (i) contacting the sample with a first binding agent which binds to an epigenetic feature of a cell free nucleosome; (ii) contacting the sample bound by the first binding agent in step (i) with a second binding agent which binds to cell free nucleosomes; and (iii) detecting or quantifying the binding of the second binding agent in the sample.

12. The method of claim 1, wherein the method of detection or measurement comprises: (i) contacting the sample with a first binding agent which binds to cell free nucleosomes; (ii) contacting the sample bound by the first binding agent in step (i) with a second binding agent which binds an epigenetic feature of a cell free nucleosome; and (iii) detecting or quantifying the binding of the second binding agent in the sample.

13. The method of claim 1, wherein the subject is a human or an animal subject.

14. The method of claim 1, wherein the method is repeated on one or more occasions and any changes in the level of cell free nucleosomes or component thereof is used to monitor the progression of multiple sclerosis in the subject.

15. The method of claim 1, further comprising comparing the level of cell free nucleosomes or component thereof in the body fluid sample of the subject with one or more controls.

16. The method of claim 15, wherein the one or more controls is a healthy subject.

17. The method of claim 1, wherein the level of cell free nucleosomes or component thereof is elevated compared to a control.

18. The method of claim 1, wherein the level of cell free nucleosomes is detected or measured as one of a panel of measurements.

19-21. (canceled)

22. A method of detecting a subject in need of medical treatment for multiple sclerosis, comprising:
(i) contacting a body fluid sample obtained from the subject with a binding agent to detect or measure the level of cell free nucleosomes or a component thereof; and
(ii) using the level of cell free nucleosomes as an indicator that the subject is in need of medical treatment for multiple sclerosis.

23-25. (canceled)

26. A method of treating a subject suffering from multiple sclerosis, comprising:
(i) contacting a body fluid sample obtained from the subject with a binding agent to measure the level of cell free nucleosomes or a component thereof;
(ii) using the level of cell free nucleosomes or component thereof measured in step (i) as indicative of the presence and/or severity of multiple sclerosis in the subject; and
(iii) administering a treatment to the subject identified in need of treatment in step (ii).
